(19) United States
(12) Patent Application Publication
(10) Pub. No.: US 2003/0026817 A1
(43) Pub. Date: Feb. 6, 2003

(54) Magnetic body care compositions

(76) Inventors: Daniel Brehmer, Santa Barbara, CA (US); Giovanni James Guidotti, Redondo Beach, CA (US); Arthur Charles Guidotti, Sherman Oaks, CA (US)

Correspondence Address: DOWELL & DOWELL PC, SUITE 309, 1215 JEFFERSON DAVIS HIGHWAY, ARLINGTON, VA, 22202

(21) Appl. No.: 10/033,896

(22) Filed: Jan. 3, 2002

Related U.S. Application Data
(60) Provisional application No. 60/259,230, filed on Jan. 3, 2001.

(51) Int. Cl. A61K 6/00

(52) U.S. Cl. 424/401

(57) ABSTRACT

Cosmetic and pharmaceutical compositions for topical application which include a liquid carrier, a magnetic metalloprotein and magnetic particles.

### CROSS REFERENCE TO RELATED APPLICATION

This application claims the benefit of U.S. Provisional Patent Application filed on Jan. 3, 2001 and assigned Serial No. 60/259,230 entitled MAGNETIC BODY CARE COMPOSITIONS with inventors, Daniel Brehmer, Giovanni James Guidotti and Arthur Charles Guidotti.

### BACKGROUND OF THE INVENTION

1. Field of the Invention

The present invention relates to body care compositions including both pharmaceutical and cosmetic compositions such as shampoos, hair conditioners, moisturizers, lotions, body creams and the like which are magnetically enhanced by using combinations of magnetite and metalloproteins.

2. Brief Discussion of the Related Art

There have been numerous innovative products recently developed to stimulate biological effects on individuals through the application or exposure to magnetic fields. Many such developments include the exposure of a body or parts of a body directly to magnetic fields developed by permanent or electromagnets physically retained in products which are worn, carried by or which are designed to be used in close proximity of an individual. By way of example, magnetic inserts have been developed to be carried by wearing apparel such that a magnetic field is applied directly to a particular area of an individual's body in order to stimulate blood circulation.

As the benefits of magnetic fields have been more widely accepted, the use of magnetic fields has expanded to the area of cosmetics and pharmaceutical products. In U.S. Pat. No. 6,333,655 to Lahanas et al., magnetic cosmetic compositions are disclosed which incorporate cosmetic and pharmaceutical compositions which include magnetic particles bound to iron exchange resin beads. As discussed in the patent, such cosmetic compositions provide benefits such as in enhancing the moisturizing of skin by the application of a cosmetic or pharmaceutical composition containing magnetic particles including magnetic iron metal bound to the iron exchange resin beads. The patent also notes other uses of magnetic particles in topically applied cosmetic preparations as disclosed in WO publication 9503061, Japanese Patent JP5309016 and German Patent DE3629761.

As also discussed in the patent to Lahanas et al., it has been determined that including magnetic particles in a cosmetic or pharmaceutical composition can have beneficial effect on the stabilization of the cosmetic composition itself, thereby providing an additional benefit.

### SUMMARY OF THE INVENTION

The present invention is directed to cosmetic and pharmaceutical compositions or superparamagnetic suspensions which are topically applied and which include as a portion of each composition magnetite particles and metalloproteins.

The magnetite is a naturally occurring mineral which, in a pure state, has been approved for use in topically applied compositions. With the present invention, magnetite powder of high purity and approved for use under regulations of the FDA, 21 CFR §73.2250, are added to a cosmetic solution or composition and evenly distributed throughout the composition. Also blended within the composition are metalloprotein molecules which are introduced preferably from lactoferrin which is a food-grade milk product. During blending of the cosmetic and pharmaceutical compositions of the present invention, the magnetite and the lactoferrin are evenly mixed throughout the solutions or compositions such that the metalloprotein molecules delivered by the lactoferrin are exposed to the magnetic field created by the magnetite particles. The exposure of the lactoferrin metal protein molecules polarizes the metalloprotein molecules along the lines of the magnetic field of the particles.

To further enhance the magnetization of the particles as well as the metalloproteins, the mixing of the solutions or compositions of the present invention are performed in mixing containers to which magnetic fields are applied such that all constituents within the mixing containers are subjected to magnetic fields of permanent or electromagnetic magnets mounted to the containers.

In a preferred embodiment of the present invention, microscopic particles of magnetite and magnetic metalloproteins are blended with shampoos and conditioners in order to deliver magnetic fields to the area of an individual's or animal's hair and scalp. The metalloproteins align with the magnetic fields of the magnetite in order to provide an exchange of magnetic energy such that the shampoos or conditioners including the magnetic particles of the present invention develop a myriad of microscopic domains which immerse each portion of the hair and scalp in fields of varying magnetic energy.

It is the primary object of the present invention to provide topically applied cosmetic and pharmaceutical compositions and solutions with magnetic particles including magnetite and magnetic metalloproteins which are uniformly mixed throughout the compositions and solutions in such a manner that the magnetic metalloproteins are aligned to create magnetic fields at areas of skin, hair and scalp to which such compositions are applied.

It is also an object of the present invention to allow numerous cosmetic and pharmaceutical compositions which are to be topically applied to benefit from magnetic affects of magnetic particles wherein magnetic particles magnetize metalloproteins which are beneficial to conditioning and treatment of hair and skin, including the scalp of an individual or animal.

### BRIEF DESCRIPTION OF THE DRAWINGS

A better understanding of the invention will be had with respect to the following drawings wherein:

FIG. 1 is an illustrative view of a metalloprotein showing how the metalloprotein is generally structured;

FIG. 2 is an illustrational view showing the interaction of a plurality of magnetic metalloproteins with a magnetic crystal or particle such as a magnetite;

FIG. 3 is an illustrational view showing the magnet metalloproteins and magnetite establishing microscopic domains of magnetization relative to a strand of hair;

FIG. 4 is a front perspective view of a mixing container showing one manner in which the container is magnetized so that compositions being mixed within the container are subject to a magnetic field in accordance with the teachings of the present invention;

FIG. 5 is a top plan view of another embodiment of magnetized mixing container in accordance with the invention;

FIG. 6 is a partial cross sectional view taken along line 6-6 of FIG. 5; and

FIG. 7 is a partial top plan view of another magnetized mixer in accordance with the invention.

### DESCRIPTION OF THE PREFERRED EMBODIMENT

The present invention is generally directed to magnetic cosmetic and pharmaceutical compositions wherein the magnetic affect is achieved by combining magnetic metalloproteins with magnetic particles such as magnetite in such compositions. The inventions will be described more specifically for use in shampoos and hair conditioners, however, the magnetic compositions of the present invention may be used with substantially any topically applied cosmetic or pharmaceutical composition including hair coloring preparations, body creams and lotions, skin and face creams and lotions, styling gels, soaps, dispersions, oil-in-oil or oil-in-water emulsions, suspensions, foams, mousses, and the like. The compositions of the present invention may be used with current pharmaceutical or cosmetic products in order to provide the benefit of magnetic field application to specific areas of an individual's or animal's body without the compositions of the present invention adversely affecting the quality, shelf life, or other beneficial affect of such products.

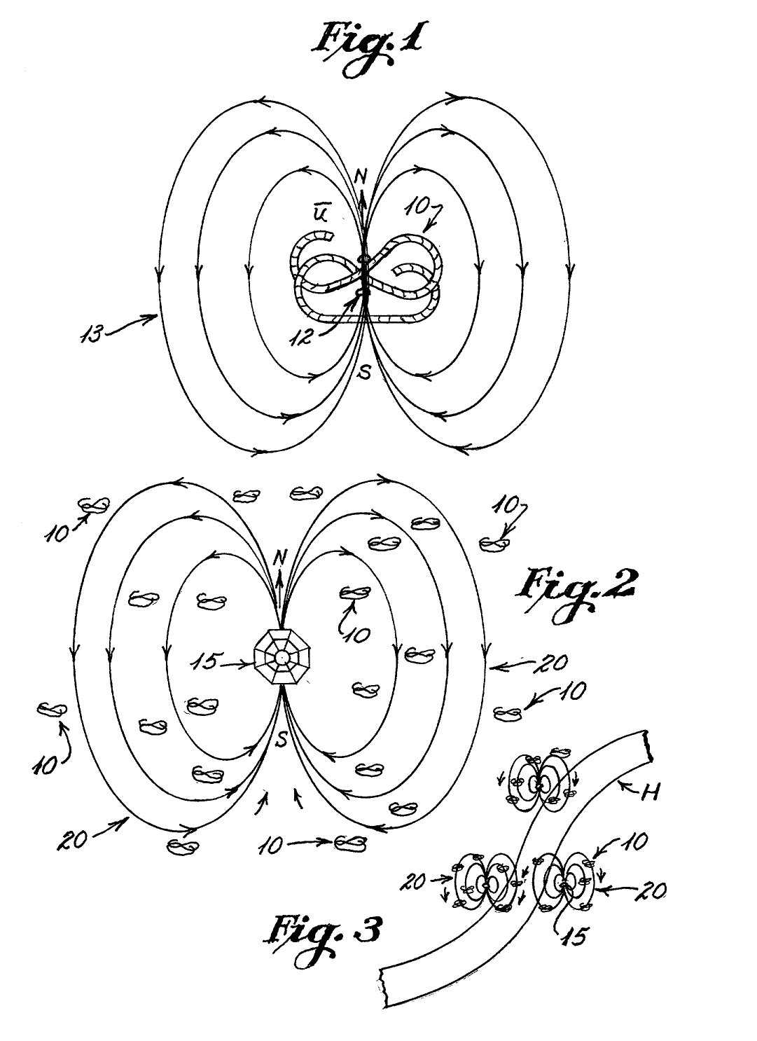

With specific reference to FIG. 1, the present invention includes compositions having therein magnetic metalloproteins. The proteins are generally shown at 10. The protein surrounds or is wrapped around a magnetic ion 12 which exhibits a magnetic moment \overline{u} such that a magnetic field 13 is established as shown in FIG. 1 from north to south. Such a magnetic metalloprotein is obtained in the preferred embodiment from food-grade lactoferrin which is derived from milk. A suitable lactoferrin is available from Agricell, their product description Bovine Lactoferrin.

The magnetic particles associated with the magnetic metalloproteins are fact miniature or microscopic magnets of approximately one micron in size. The magnetic particles create microscopic domains of magnetization wherein, within each domain, the magnetic moment of the metalloprotein is aligned with the magnetic field of the magnetic particle associated therewith.

In accordance with the teachings of the present invention, the compositions of the present invention also include magnetite (Fe₃O₄) particles 15. The magnetite may be provided as a black iron oxide pigment of high purity grade such as HP, EP and AP grades which are acceptable under FDA guideline 21 CFR §2250 for use in cosmetic applications.

With specific reference to FIG. 2, an illustration is shown of the magnetic metalloproteins 10 aligning with magnetic field 20 of the magnetite particles 15 in the composition whereby they are aligned with the magnetic field.

With specific reference to FIG. 3, when the compositions of the present invention are uniformly blended throughout a cosmetic or pharmaceutical compositions and applied to an individual's scalp, such as by applying a shampoo or conditioner to hair “H”, the proteins which are beneficial to the hair particles are also magnetized and thus provide small microscopic areas of magnetization along the surface of the hair thus providing not only the benefit of the protein but of the magnetic field created about the protein.

In use, the weight percent of the magnetic metalloprotein in the final composition should be in the range of 0.001% to 0.01% with the percentage of particles of magnetite being present in the amount by weight of between approximately 0.1% to 0.25%.

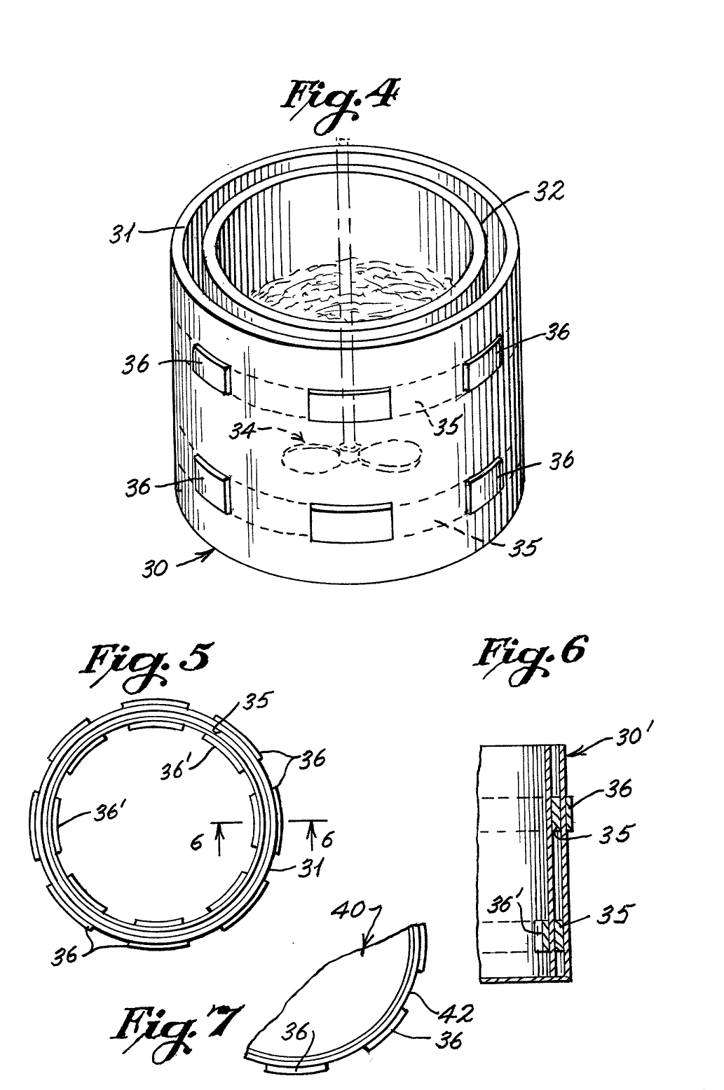

With specific reference to FIG. 4, there is disclosed a mixing vessel 30 constructed of stainless steel or other non-magnetizable material having two spaced generally circular side walls 31 and 32. An agitator 34 is shown in dotted line extending into the top of the vessel and is provided with a mixing blade for purposes of mixing a solution or slurry within the vessel. In order to magnetize particles within the suspension or slurry being mixed within the vessel or container, bands of magnetizable steel 35 are be applied between the inner and outer side walls of the mixing vessel. Mounted in alignment with the bands of steel are either electro or permanent magnets 36. A plurality of magnets 36 are shown provided along each band 35 along the outer side wall 31 in FIG. 4, however, the magnets could be positioned along the inside of wall 32.

During mixing, the magnets will create a magnetic field within the mixing vessel which magnetic field will affect the magnetic particles within the compositions of the present invention as they are being blended.

With respect to FIGS. 5 and 6, another embodiment of a mixing vessel 30′ is disclosed wherein the vessel is provided with a plurality of magnets 36 and 35′ mounted along alternating bands 35 between the side walls.

With reference to FIG. 7, another mixing container 40 is shown having a single circular side wall 40 with bands of magnetizable steel 42 being mounted exteriorly thereof. In this embodiment, the magnets 36 are applied directly to the bands 42.

The foregoing description of the preferred embodiment of the invention has been presented to illustrate the principles of the invention and not to limit the invention to the particular embodiment illustrated. It is intended that the scope of the invention be defined by all of the embodiments encompassed within the following claims and their equivalents.

We claim:

1. Cosmetic and pharmaceutical compositions for developing a beneficial magnetic field when applied topically to a specific area of a mammal's hair or skin which includes a liquid carrier, a magnetic metalloprotein and magnetic particles.

2. The cosmetic and pharmaceutical compositions of claim 1 in which the magnetic particles are formed of magnetite.

3. The composition of claim 2 in which the liquid carrier is selected from a group of carriers consisting of creams, lotions, jells, soaps, dispersions, oil-in-oil emulsions, oil-in-oil water emulsions, suspensions, foams and mousses.

4. The composition of claim 1 in which the magnetic metalloprotein is derived from a food-grade lactoferrin.

5. The cosmetic and pharmaceutical compositions of claim 2 when the magnetic particles are generally not greater than 1 micron in size.

6. The composition of claim 2 in which the magnetic metalloprotein in the compositions is by weight in the range of 0.001% to 0.01% with the range by weight percentage of particles of magnetite being present in the composition between 0.1% to 0.25%.

7. A method of developing magnetic effects by topical application to hair and skin of a mammal consisting of blending a composition including a liquid carrier, a magnetic metalloprotein and magnetic particles, and thereafter applying the composition to the hair or skin of the mammal.

8. A method of mixing a topically applied composition for cosmetic or pharmaceutical use wherein the composition includes a liquid carrier, magnetic metalloprotein and magnetic particles, the method including providing a non-magnetizable material container and introducing the composition into the container, agitating the composition in the container while applying a magnetic field to the composition within the container.

9. A mixing apparatus for blending a cosmetic or pharmaceutical topical composition including a liquid carrier, a metalloprotein and magnetic particles, the apparatus including a vessel having a side and bottom walls formed of a non-magnetizable material, and means mounted to said vessel for mounting magnetic field developing elements thereto for purposes of generating a magnetic field within the vessel.

10. The container of the claim 9 in which the means for mounting includes at least one magnetizable material band secured to one of said bottom or said side walls of said vessel.
